(19) United States
(12) Patent Application Publication
(10) Pub. No.: US 2014/0271749 A1
(43) Pub. Date: Sep. 18, 2014

(54) Silica Materials for Reducing Oral Malodor

(71) Applicant: J.M. Huber Corporation, Atlanta, GA (US)

(72) Inventors: Karl W. Gallis, Perryville, MD (US); Terry W. Nassivera, North East, MD (US); William H. Pitcock, JR., North East, MD (US)

(73) Assignee: J.M. Huber Corporation, Atlanta, GA (US)

(21) Appl. No.: 14/289,717

(22) Filed: May 29, 2014

Related U.S. Application Data
(63) Continuation of application No. 13/026,864, filed on Feb. 14, 2011.
(60) Provisional application No. 61/305,998, filed on Feb. 19, 2010.

(51) Int. Cl. A61K 8/25; A61Q 11/00; A61K 8/02

(52) U.S. Cl. CPC A61K 8/25; A61K 8/0241; A61Q 11/00
USPC 424/401; 424/49; 423/339; 428/402

(57) ABSTRACT

Disclosed herein are silica materials comprising an adduct on at least a portion of a surface thereof. The silica materials are capable of binding volatile sulfur compounds (VSC)s and thereby reducing oral malodor. Also disclosed are dentifrice compositions, oral care compositions comprising the silica materials, and methods of making and using the silica materials.

### CROSS-REFERENCE TO RELATED APPLICATIONS

This application claims the benefit of priority of U.S. Provisional Application 61/305,998, filed Feb. 19, 2010, the entire contents of which are incorporated herein by reference.

### BACKGROUND

Bacterial putrefaction in the oral cavity of the mouth can result in the formation of foul smelling volatile sulfur compounds (VSC)s that contribute to oral malodor, or the offensive odor known as bad breadth. VSCs such as hydrogen sulfide, methyl mercaptan, and dimethyl sulfide are believed to be the most common sources of putrid odors originating from the mouth. VSC production occurs in the oral cavity when microbial flora shift from predominately gram-positive to predominately gram-negative anaerobic flora.

The shift to gram-negative bacteria is typically accompanied by a depletion of carbohydrates and a rise in the pH level of saliva of from below about 6.5 to about 7.2. As carbohydrates are depleted, gram-positive flora begin to lose fuel sources and are thus suppressed, while gram-negative flora become progressively more dominant. Gram-negative flora, unlike gram-positive flora, are capable of breaking down proteins into short peptides and amino acids by proteolysis. Certain proteins, such as those containing cysteine and methionine residues, can be further broken down into VSCs.

Solutions for oral malodor include mouthwash, chewing gums, breath strips, lozenges, mints, and toothpastes. Unfortunately, many of these products are incapable of reducing or preventing oral malodor for an extended period of time. Additionally, some of these products do little to prevent the exhalation of putrid VSCs. A need therefore exists for new compositions and methods for reducing oral malodor. These needs and other needs are satisfied by the present invention.

### SUMMARY

Disclosed herein are silica materials comprising an adduct on at least a portion of a surface thereof. The silica materials are capable of binding volatile sulfur compounds (VSC)s and thereby reducing oral malodor. Also disclosed are dentifrice compositions, oral care compositions comprising the silica materials, and methods of making and using the silica materials.

The advantages of the invention will be set forth in part in the description which follows, and in part will be obvious from the description, or can be learned by practice of the aspects described below. The advantages described below will be realized and attained by means of the elements and combinations particularly pointed out in the description which follows. It is to be understood that both the foregoing general description and the following detailed description are exemplary and explanatory only and are not restrictive.

### BRIEF DESCRIPTION OF THE DRAWINGS

FIG. 1 is a diagram of the testing apparatus used to measure mercaptan absorption using the disclosed silica materials.

FIG. 2 is a plot of methyl mercaptan peak area vs. time obtained using the Comparative Example. ♦ represents percent remaining and ▪ represents percent reduction.

FIG. 3 is a plot obtained from a repeated time study using the Comparative Example, which includes an outlier point. The plot shows methyl mercaptan peak area vs. time; ♦ represents percent remaining and ▪ represents percent reduction.

FIG. 4 is a plot from the repeated time study using the Comparative Example, which does not include the outlier point. The plot shows methyl mercaptan peak area vs. time; ♦ represents percent remaining and ▪ represents percent reduction.

FIG. 5 is a plot of methyl mercaptan peak area vs. time obtained using Example 1A. ♦ represents percent remaining and ▪ represents percent reduction.

FIG. 6 is a plot from a repeated time study using Example 1A. The plot shows methyl mercaptan peak area vs. time. ♦ represents percent remaining and ▪ represents percent reduction.

FIG. 7 is a plot showing methyl mercaptan peak area vs. time obtained using Example 1B. ♦ represents percent remaining and ▪ represents percent reduction.

FIG. 8 is a plot showing methyl mercaptan peak area vs. time obtained using Example 1C. ♦ represents percent remaining and ▪ represents percent reduction.

FIG. 9 is a plot showing methyl mercaptan peak area vs. time obtained using Example 1D. ♦ represents percent remaining and ▪ represents percent reduction.

FIG. 10 is a plot showing methyl mercaptan peak area vs. time obtained using Example 1E. ♦ represents percent remaining and ▪ represents percent reduction.

FIG. 11 is a plot showing headspace recoveries in the multi-dosing study for Examples 1A-E and Comparative Example, described below. The headspace recoveries are as follows: ▪—Example 1A; ♦—Example 1B; ▴—Example 1C; X—Example 1D; [symbol] —Comparative Example; +—Example 1E.

FIG. 12 is a plot of soluble concentration versus pH for Examples 1A-E. The samples are as follows: ▪—Example 1A; ♦—Example 1B; ▴—Example 1C; X—Example 1D; +—Example 1E.

### DETAILED DESCRIPTION

Before the present compounds, compositions, composites, articles, devices and/or methods are disclosed and described, it is to be understood that the aspects described below are not limited to specific compounds, compositions, composites, articles, devices, methods, or uses as such may, of course, vary. It is also to be understood that the terminology used herein is for the purpose of describing particular aspects only and is not intended to be limiting.

In this specification, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification, the singular forms “a,” “an” and “the” include plural referents unless the context clearly dictates otherwise. Thus, for example, reference to “a silica material” includes mixtures of two or more such materials, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different silica particles and adducts are disclosed and discussed, each and every combination and permutation of the silica particle and adduct are specifically contemplated unless specifically indicated to the contrary. If a class of adducts A, B, and C are disclosed as well as a class of adducts D, E, and F and an example of a combination adduct, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

As used herein, “mean particle size” refers to the particle size for which 50% of the sample by number has a smaller size and 50% of the sample by number has a larger size.

The present invention relates to silica materials that comprise an adduct, such as a transition metal, which are capable of absorbing VSCs and are thus useful for reducing oral malodor. The silica materials of the invention can be retained in the oral cavity of the mouth, which may enable VSC absorption over an extended period of time.

In one aspect, the silica material has a mean particle size of from 1 to 15 microns and has an adduct present on at least a portion of its surface. The adduct is present on the surface of the silica particles in the silica material such that the adduct is available for interaction with VSCs, i.e., the adduct is exposed.

In a further aspect, a slurry of the silica material exhibits at least a 35% reduction, 45% reduction, or 50% reduction, in headspace in the VSC absorption test, i.e., the lest described below in the Examples section under the heading “VSC Absorption Test” and “Multi-dosing Study.” The specific test for determining % headspace reduction (also referred to as “recovery”) is the aqueous test described below under the heading “Multi-dosing Study.”

Generally, the silica material of the invention has a mean particle size of 1 to 15 microns. In a further aspect, the silica material has a mean particle size of greater than 5 microns to 15 microns, or for example, from 8 to 15 microns. Preferably, less than about 2% of the silica material of the invention has a particle size of greater than 44 microns.

The adduct present on the silica material is preferably a metal adduct, such as a transition metal or post-transition metal. Non-limiting examples of suitable metal adducts include zinc, tin, copper, strontium, aluminum, and mixtures thereof. The silica material is prepared by the addition of the adduct in the form of a water-soluble metal salt during the formation of precipitated silica material. Any metal salt that is soluble in acidic conditions would be suitable, such as metal nitrates, metal chlorides, metal sulfates, and the like.

In a further aspect, the silica material of the invention has a mean particle size of 1 to 15 microns and has a first and second adduct present on at least a portion of its surface, wherein the first and second adducts are different. Such a silica material can have a mean particle size of 1 to 5 microns, from greater than 5 microns to 15 microns, or from 8 to 15 microns. Examples of such silica materials include without limitation those having aluminum and zinc, aluminum and copper, zinc and tin, strontium and copper, or tin and strontium adducts both present on at least a portion of the surface of the silica.

In a still further aspect, the silica material of the invention has a mean particle size of 1 to 15 microns and has an adduct present on at least a portion of its surface, wherein the adduct is present in an amount ranging from 0.025% to 10% by weight relative to the silica material as a whole, i.e., metal adduct weight/(metal adduct weight+silica weight). In a further aspect, the adduct is present in an amount ranging from 0.025% to 5%, from 0.025% to 3%, from 0.025% to 2%, or from 0.025% to 1% by weight relative to the silica material. In further aspects, the adduct is present in an amount ranging from 0.1% to 10%, 0.35% to 5%, or 1% to 3% by weight of the silica material.

The silica material can include a variety of types of silicas, including precipitated silicas, fumed silica, silica gels, and the like. In one particular aspect, the silica material contains a precipitated silica and is prepared according to the following process. An aqueous solution of an alkali silicate, such as sodium silicate, is charged into a reactor equipped with mixing means adequate to ensure a homogeneous mixture. The alkali silicate solution in the reactor is preheated to a temperature of between about ambient temperature and 130° C., preferably from about 50 to 100° C., and more preferably from about 65° C. and about 100° C., or from about 60° C. to about 90° C. The alkali silicate solution can have an alkali silicate concentration of approximately 8.0 to 35 wt %, such as from about 8.0 to about 20 wt %. The alkali silicate can be a sodium silicate with a SiO₂:Na₂O ratio of from about 1 to about 3.5, such as about 2.4 to about 3.4. The quantity of alkali silicate charged into the reactor is about 5 wt % to 100 wt % of the total silicate used in the batch. Optionally, an electrolyte, such as sodium sulfate solution or aluminum sulfate (Alum) solution, can be added to the reaction medium. Additionally, this mixing can be performed under high-shear conditions if desired.

To the reactor is then simultaneously added: (1) an aqueous solution of an acidulating agent or acid, such as sulfuric acid; (2) additional amounts of an aqueous solution containing the same species of alkali silicate as is in the reactor, such aqueous solution being preheated to a temperature of about 65° C. to about 100° C. An adduct compound is added to the acidulating agent solution prior to the introduction of the acidulating agent solution into the reactor. The adduct compound is premixed with the acidulating agent solution in a concentration of mol. of adduct compound to L of acidulating agent solution of about 0.002 to about 0.185, preferably about 0.074 to about 0.150. Optionally, if higher adduct concentrations are required in the adduct-treated precipitated silica material, an aqueous solution of the adduct compound can be used in place of the acid.

The adduct compound can be a variety of metal compounds, including metal salts such as zinc(II) salts, tin(II) salts, iron(III) salts, copper(II) salts, and Alum. Specific examples include zinc(H) chloride, tin(II) chloride, iron(III) nitrate, copper(II) nitrate, and hydrated aluminum potassium sulfate.

The acidulating agent solution preferably has a concentration of acidulating agent of about 6 to 35 wt %, such as about 9.0 to about 20 wt %. After a period of time the inflow of the alkali silicate solution is stopped and the acidulating agent solution is allowed to flow until the desired pH is reached.

The reactor batch is allowed to age or “digest” for between 5 minutes to 30 minutes at a set digestion temperature, with the reactor batch being maintained at a constant pH. After the completion of digestion, the reaction batch is filtered and washed with water to remove excess by-product inorganic salts until the wash water from the silica filter cake obtains a conductivity of less than about 2000 μmhos. Because the conductivity of the silica filtrate is proportional to the inorganic salt by-product concentration in the filter cake, then by maintaining the conductivity of the filtrate to be less than 2000 μmhos, the desired low concentration of inorganic salts, such as Na₂SO₄ in the filter cake can be obtained. The silica filter cake is slurried in water, and then dried by any conventional drying techniques, such as spray drying, to produce adduct-treated precipitated silica material containing from about 3 wt % to about 50 wt % of moisture. The adduct-treated precipitated silica material can then be milled to obtain the desired particle size of between about 1 μm to 15 μm, from 1 μm to 5 μm, from greater than 5 μm to 15 μm, or from 8 μm to 15 μm.

The present invention also relates to dentifrices comprising the disclosed silica materials. As used herein, a “dentifrice composition” refers to a composition that can be used to maintain oral hygiene, for example by cleaning accessible surfaces of the teeth. Examples include toothpastes, liquid dentifrices, paste dentifrices, powdered dentifrices, and the like.

Examples of dentifrices are those that, in addition to the silica material of the invention, comprise water, detergent, humectant, binder, flavoring agents, powdered abrasive, or combinations thereof as the ingredients. Dentifrice formulations can also comprise ingredients which must be dissolved prior to incorporation into the dentifrice formulation (e.g. anti-caries agents such as sodium fluoride, sodium phosphates, flavoring agents such as saccharin).

The silica material of the invention can be present in the dentifrice composition in an amount generally ranging from 0.01 to 50%, from 0.01 to 30%, or from 0.01 to 25% by weight relative to the entire dentifrice composition. If the silica material of the invention is abrasive in nature, the amount can be from 0.05 to about 15% by weight. If the adduct-treated precipitated silica material is a viscosity modifier (thickening agent), the amount can be from 0.05 to about 10% by weight. However, the silica material of the invention can be present as an agent useful for reducing oral malodor. As such, the amount may be within the range noted above within the dentifrice formulation, but the materials may not provide any appreciable degree of thickening or abrasivity to the dentifrice, but will provide oral malodor reduction benefits. Such formulations may also include potassium nitrate salts, as one example, of a suitable other desensitizing material, if desired.

In a further aspect, the dentifrice composition comprises at least one other component such as an abrasive other than the adduct-treated precipitated silica material, at least one thickening agent other than the adduct-treated precipitated silica material, at least one solvent, at least one preservative, at least one surfactant, or a combination thereof; wherein the silica material of the invention is present as an abrasive agent, thickening agent, or both, within the dentifrice.

In one aspect, the disclosed silica materials can be utilized alone as the abrasive in the dentifrice composition, or as an additive or co-abrasive with other abrasive materials discussed herein or known in the art. Any number of other conventional types of abrasive additives can be present within the dentifrice compositions of the invention. Other such abrasive particles include, for example, precipitated calcium carbonate (PCC), ground calcium carbonate (GCC), chalk, bentonite, dicalcium phosphate or its dihydrate forms, silica gel (by itself, and of any structure), precipitated silica, amorphous precipitated silica (by itself, and of any structure as well), perlite, titanium dioxide, dicalcium phosphate, calcium pyrophosphate, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. Such materials can be introduced into the dentifrice compositions to tailor the polishing characteristics of the target formulation.

In addition to the abrasive component, the dentifrice can also contain one or more organoleptic enhancing agents. Organoleptic enhancing agents include humectants, sweeteners, surfactants, flavorants, colorants and thickening agents, (also sometimes known as binders, gums, or stabilizing agents).

Humectants serve to add body or “mouth texture” to a dentifrice as well as preventing the dentifrice from drying out. Suitable humectants include polyethylene glycol (at a variety of different molecular weights), propylene glycol, glycerin (glycerol), erythritol, xylitol, sorbitol, mannitol, lactitol, and hydrogenated starch hydrolyzates, and mixtures thereof. In specific examples, humectants are present in an amount from about 20 wt % to about 50 wt % of the dentifrice composition, for example 40 weight %.

Sweeteners can be added to the dentifrice composition (e.g., toothpaste) to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, and glucose.

Surfactants can be used in the dentifrice compositions of the invention to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. Sodium lauryl sulfate is a preferred surfactant. The surfactant is typically present in the oral care compositions of the present invention in an amount of about 0.1 to about 15% by weight, preferably about 0.3% to about 5% by weight, such as from about 0.3% to about 2.5%, by weight.

Flavoring agents can also be added to dentifrice compositions. Suitable flavoring agents include, but are not limited to, oil of wintergreen, oil of peppermint, oil of spear, lint, oil of sassafras, and oil of clove, cinnamon, anethole, menthol, thymol, eugenol, eucalyptol, lemon, orange and other such flavor compounds to add fruit notes, spice notes, etc. These flavoring agents generally comprise mixtures of aldehydes, ketones, esters, phenols, acids, and aliphatic, aromatic and other alcohols.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Thickening agents are useful in the dentifrice compositions to provide a gelatinous structure that stabilizes the toothpaste against phase separation. Suitable thickening agents include silica thickener; starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof. Typical levels of thickening agents or binders are from about 0 wt % to about 15 wt % of a toothpaste composition.

Useful silica thickeners for utilization within a toothpaste composition, for example, include, as a non-limiting example, an amorphous precipitated silica such as ZEODENT 165 silica. Other preferred (though non-limiting) silica thickeners are ZEODENT 153, 163 and/or 167 and ZEOFREE, 177, and/or 265 silicas, all available from J. M. Huber Corporation.

Therapeutic agents can also be used in the compositions to provide for the prevention and treatment of dental caries, periodontal disease and temperature sensitivity. Examples of therapeutic agents, without intending to be limiting, are fluoride sources, such as sodium fluoride, sodium monofluorophosphate, potassium monofluorophosphate, stannous fluoride, potassium fluoride, sodium fluorosilicate, ammonium fluorosilicate and the like; condensed phosphates such as tetrasodium pyrophosphate, tetrapotassium pyrophosphate, disodium dihydrogen pyrophosphate, trisodium monohydrogen pyrophosphate; tripolyphosphates, hexametaphosphates, trimetaphosphates and pyrophosphates; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tannase, and proteases; quaternary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; peroxides and the like. Therapeutic agents may be used in dentifrice formulations singly or in combination at a therapeutically safe and effective level.

Preservatives can also be added to the compositions of the present invention to prevent bacterial growth. Suitable preservatives approved for use in oral compositions such as methylparaben, propylparaben and sodium benzoate can be added in safe and effective amounts.

The dentifrices disclosed herein can also contain a variety of additional ingredients such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pII control agents, oxidizing agents, antioxidants, and the like.

Water typically provides the balance of the composition in addition to the additives mentioned above. The water is preferably deionized and free of impurities. The dentifrice will usually comprise from about 5 wt % to about 70 wt % of water, for example 5 wt % to 35 wt %, such as 11 wt % water.

The silica materials of the invention can also be incorporated into a variety of other oral care compositions, including breath strips, gums, such as chewing gums, mouthwashes, mouth rinses, confections (e.g., lozenges, pressed tablets, hard candies, etc.), edible films, mouthsprays, and teeth whitening strips.

Also disclosed are methods for reducing the amount of VSCs, such as mercaptan, in an atmosphere that contains VSCs, such as an oral cavity, by contacting the VSC-containing atmosphere with a disclosed silica material or dentifrice composition, thereby reducing the level of VSCs in the atmosphere. A specific example is a method of reducing oral malodor in a subject, such as a human, by administering the silica material or dentifrice to the oral cavity of the subject, thereby reducing oral malodor in the subject. As discussed above, the disclosed materials and dentifrices are effective at reducing VSC levels in an atmosphere (see Examples below), and thus are effective at reducing oral malodor given that VSCs cause oral malodor. In one aspect, the subject (e.g., human), which accepts the silica material or the dentifrice of the invention has a need for treatment, i.e., the subject has oral malodor. In other aspects, the silica material or dentifrice can be used for prophylactic treatment, i.e., given to a subject such as a human that does not necessarily suffer from oral malodor at the time of administration.

To test for a reduction in oral malodor in a subject, the test below under the heading “Mutt-dosing Study in Aqueous Conditions” can be used to show such a reduction, i.e., using a vapor sample collected from the oral cavity of the subject. Alternatively, known methods in the art, such as smell tests wherein oral cavity vapor samples from the subject are evaluated before and after administration, can also be used to show a reduction in oral malodor.

In a further aspect, disclosed is a method for reducing oral malodor, comprising the steps of a) providing a composition suitable for oral administration comprising the disclosed silica material; and b) administering the composition to a subject having the presence of VSCs in their mouth, thereby allowing the silica material of the composition to absorb the VSCs and in turn reduce the subject's oral malodor.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Silica Materials

### Example 1A

410 mL of silicate (13.3%, 1.112 g/mL, 3.32 MR) was added to a 2-gallon reactor and heated to 85° C. with stirring at 300 RPM. Silicate (13.3%, 1.112 g/mL, 3.32 MR) and a sulfuric acid/zinc(II) chloride solution (11.4%, 1.078 g/mL sulfuric acid containing 0.077 mol ZnCl₂/L acid) were then simultaneously added at 82.4 mL/min and 24.8 mL/min, respectively, for 47 minutes. After 47 minutes, the flow of silicate was stopped, and the pH was adjusted to 5.5 with continued flow of acid. Once pH 5.5 was reached, the batch was allowed to digest for 10 minutes at 90° C. and was then dropped out of the reactor. It was filtered and washed with two displacements of deionized water. The sample was dried at 105° C. overnight. The above batch was made in triplicate, combined, and air-milled to an average particle size of about 3.0 μm. Material from Example 1A was used to obtain FIGS. 5 and 6.

### Example 1B

410 mL of silicate (13.3%, 1.112 g/mL, 3.32 MR) was added to a 2-gallon reactor and heated to 85° C. with stirring at 300 RPM. Silicate (13.3%, 1.112 g/mL, 3.32 MR) and a sulfuric acid/tin(II) chloride solution (11.4%, 1.078 g/mL sulfuric acid containing 0.042 mol SnCl₂.2H₂O/L acid) were then simultaneously added at 82.4 mL/min and 24.8 mL/min, respectively, for 47 minutes. After 47 minutes, the flow of silicate was stopped, and the pH was adjusted to 5.5 with continued flow of acid. Once pH 5.5 was reached, the batch was allowed to digest for 10 minutes at 90° C. and was then dropped out of the reactor. It was filtered and washed with two displacements of deionized water. The sample was dried at 105° C. overnight. The above batch was made in triplicate, combined, and air-milled to an average particle size of about 3.0 μm. Material from Example 1B was used to obtain FIG. 7.

### Example 1C

410 mL of silicate (13.3%, 1.112 g/mL, 3.32 MR) was added to a 2-gallon reactor and heated to 85° C. with stirring at 300 RPM. Silicate (13.3%, 1.112 g/mL, 3.32 MR) and a sulfuric acid/iron(III) nitrate solution (11.4%, 1.078 g/mL sulfuric acid containing 0.089 mol Fe(NO₃)₃.9H₂O/L acid) were then simultaneously added at 82.4 mL/min and 24.8 mL/min, respectively, for 47 minutes. After 47 minutes, the flow of silicate was stopped, and the pH was adjusted to 5.5 with continued flow of acid. Once a pH 5.5 was reached, the batch was allowed to digest for 10 minutes at 90° C. and was then dropped out of the reactor. It was filtered and washed with two displacements of deionized water. The sample was dried at 105° C. overnight. The above batch was made in triplicate, combined, and air-milled to an average particle size of about 3.0 μm. Material from Example 1C was used to obtain FIG. 8.

### Example 1D

410 mL of silicate (13.3%, 1.112 g/mL, 3.32 MR) was added to a 2-gallon reactor and heated to 85° C. with stirring at 300 RPM. Silicate (13.3%, 1.112 g/mL, 3.32 MR) and a sulfuric acid/copper(II) nitrate solution (11.4%, 1.078 g/mL sulfuric acid containing 0.077 mol Cu(NO₃)₂.2.5H₂O/L acid) were then simultaneously added at 82.4 mL/min and 24.8 mL/min, respectively, for 47 minutes. After 47 minutes, the flow of silicate was stopped, and the pH was adjusted to 5.5 with continued flow of acid. Once a pH 5.5 was reached, the batch was allowed to digest for 10 minutes at 90° C. and was then dropped out of the reactor. It was filtered and washed with two displacements of deionized water. The sample was dried at 105° C. overnight. The above batch was made in triplicate, combined, and air-milled to an average particle size of about 3.0 μm. Material from Example 1D was used to obtain FIG. 9.

### Example 1E

67 L of silicate (19.5%, 1.180 g/mL, 3.32 MR) and 167 L of water were added to a 400 gallon reactor and heated to 87° C. with recirculation at 30 HZ and stirring at 60 RPM. Silicate (19.5%, 1.180 g/mL, 3.32 MR) and a sulfuric acid/alum solution (17.1%, 1.12 g/mL sulfuric acid containing 0.22 mol Alum/L acid) were then simultaneously added at 12.8 L/min and 3.9 L/min, respectively, for 47 minutes. After 47 minutes, the flow of silicate was stopped, and the pH was adjusted to 5.5 with continued flow of acid. Once a pH 5.5 was reached, the batch was allowed to digest for 10 minutes and was then dropped. It was filtered and washed to a conductivity of ˜1500 μS and was spray dried. A portion of this batch was then air milled to an average particle size of ˜3.0 μm. Material from Example 1E was used to obtain FIG. 10

### Comparative Example

### Silica without Metal Adduct

410 mL of silicate (13.3%, 1.112 g/mL, 3.32 MR) was added to a 2-gallon reactor and heated to 85° C. with stirring at 300 RPM. Silicate (13.3%, 1.112 g/mL, 3.32 MR) and sulfuric acid (11.4%, 1.078 g/mL) were then simultaneously added at 82.4 mL/min and 24.8 mL/min, respectively, for 47 minutes. After 47 minutes, the flow of silicate was stopped, and the pH was adjusted to 5.5 with continued flow of acid. Once a pH 5.5 was reached, the batch was allowed to digest for 10 minutes at 90° C. and was then dropped. It was filtered and washed with two displacements of deionized water. The sample was dried at 105° C. overnight. The above batch was made in triplicate, combined, and air-milled to an average particle size of about 3.0 μm. Material from the Comparative Example was used to obtain FIGS. 2-4.

Median particle size can be determined using a Model LA-930 (or LA-300 or an equivalent) laser light scattering instrument available from Horiba Instruments, Boothwyn, Pa.

Examples 1A-1E and the Comparative Examples were analyzed for metal content by elemental analysis. The results are shown in Table 1.

TABLE 1
Metals analysis for Examples 1A-1E.

| | Al | Fe | Cu | Zn | Sn |
|---|---|---|---|---|---|
| Sample | (%) | (%) | (%) | (%) | (%) |
| Example 1A | — | — | — | 1.61 | — |
| Example 1B | — | — | — | — | 1.86 |
| Example 1C | — | 1.50 | — | — | — |
| Example 1D | — | — | 1.84 | — | — |
| Example 1E | 1.18 | — | — | — | — |
| Comparative Example | — | — | — | — | — |

Physical properties of Examples 1A-1E and the Comparative Example were also evaluated. The results are shown in Table 2.

TABLE 2
Physical properties for Examples 1A-1E.

| | AbC | Moisture | 5% |
|---|---|---|---|
| Sample | (cc/100 g) | (%) | pH |
| Example 1A | 88.0 | 3.4 | 9.1 |
| Example 1B | 86.7 | 4.6 | 9.2 |
| Example 1C | 77.4 | 5.0 | 9.4 |
| Example 1D | 95.1 | 2.8 | 9.1 |
| Example 1E | 99.5 | 6.3 | 8.8 |
| Comparative Example | 109.7 | 3.8 | 8.7 |

Water absorption values are determined with an Absorptometer “C” torque rheometer from C.W. Brabender Instruments, Inc. Approximately ⅓ of a cup of silica (or silicate) is transferred to the mixing chamber of the Absorptometer and it is mixed at 150 rpm. Water then is added at a rate of 6 ml/min, and the torque required to mix the powder is recorded. As the water is absorbed by the powder, the torque will reach a maximum as the powder transforms from a free flowing powder to a paste. The total volume of water added when the maximum torque has been achieved is then standardized to the quantity of water that can be absorbed by 100 g of powder. Since the powder is used on an as received basis (it is not previously dried), the free moisture value of the powder is used to calculate a “moisture corrected water AbC value” by the following equation.

$$Water Absorption = \frac{water absorbed ( cc ) + \% moisture}{( 100 ( g ) - \% moisture ) / 100}$$

The pH of the silica materials, including 5% pH as reported above can be determined by any conventional pH sensitive electrode.

### VSC Absorption Test

Example 1A-1E and the Comparative Example were evaluated for their ability to absorb mercaptan (CH₃SH) using the apparatus shown in FIG. 1.

The method involves evacuating a set of flasks (250-mL) in parallel and charging them with the desired test gas, in this case methyl mercaptan (CH₃SH, 1.09 ppm, balance N₂, Air Liquide America, P/N 01020002400TCL). Flask 1 contains the material of interest, flask 2 contains a charge of nitrogen (Airgas, UHPG used for purging the gas sampling syringe) and flask 3 is a blank control reference.

In a typical experiment, flask 1 was charged with 200 mg of desired silica material, and all stopcocks were fully opened. Ball valve 1 was closed, and the vacuum pump turned on. With the test gas control valve in the off position, ball valve 1 was slowly opened and the entire system evacuated for 2 minutes. After 2 minutes, ball valve 1 was closed and the test gas control valve was opened to the nitrogen gas cylinder and the system was charged with nitrogen to 2 psi. The test gas control valve was then turned to the off position and this sequence was repeated 2 more times for a total of 3 evacuations and nitrogen purges. After the third nitrogen purge, the stopcocks on flask 2 were closed, creating a nitrogen purge flask. The test gas control valve was turned to the off position, ball valve 1 was opened and the system was evacuated for another 2 minutes. At this time, the stopcocks for flasks 1 and 3 were closed on the vacuum side, followed by closure of ball valve 1. The test gas control valve was then opened to the test gas cylinder and the system charged with test gas to 2 psi. The test gas side stopcocks for flasks 1 and 3 were then closed followed by the test gas control valve. The system is now charged with test gas and evaluation can begin.

Thirty seconds after the initial test gas charge using a Gerstel 2.5-mL Headspace-HT Syringe (P/N 009980-055-00), a purge was performed using the nitrogen in flask 2. Then, 1.5 mL of headspace gas was withdrawn from flask 1 and injected onto a Hewlett Packard 5890 Series II GC oven fitted with a Restek RTX-200MS column (30 m×0.32 mm ID×1 μm df, Cat. #15654) and a 5972 Series Mass Selective Detector. The detector was running in SIM mode, scanning for m/z 48, 47, and 45. All other pertinent GC/MS information is presented in Table 3. After the initial test gas injection, the purge and withdrawal cycle was repeated 2 minutes later for the control (flask 3). This alternation was carried out for the duration of the study. These headspace concentrations can then be utilized to generate a curve of Methyl Mercaptan vs. Time and the equilibration point can be used to generate a capacity. At the end of the study, all stopcocks were opened, followed by ball valve 1 and the system was evacuated for 5 minutes.

TABLE 3
GC/MS Run Conditions

| Oven Inlet Temperature | 280° | C. |
|---|---|---|
| Oven Temperature (Isothermal) | 45° | C. |
| Gooseneck Liner | Restek P/N 20796-210.5 | |
| Column Head Pressure | 10 | psi |
| Split Flow | 30 | mL/min |

| | |
|---|---|
| Carrier Gas | Helium UHP (Airgas) |

### Mercaptan Absorption Results

Using the mercaptan absorption test described above, Examples 1A-1E and the Comparative Example were evaluated. FIGS. 2-10 show the resulting reduction curves for each respective sample over time. FIG. 1 is a diagram of the testing apparatus used to measure mercaptan absorption using the disclosed silica materials. FIG. 2 is a plot of methyl mercaptan peak area vs. time obtained using the Comparative Example. ♦ represents percent remaining and ▪ represents percent reduction. FIG. 3 is a plot obtained from a repeated time study using the Comparative Example, which includes an outlier point. The plot shows methyl mercaptan peak area vs. time; ♦ represents percent remaining and ▪ represents percent reduction. FIG. 4 is a plot from another repeated time study using the Comparative Example, which does not include the outlier point. The plot shows methyl mercaptan peak area vs. time; ♦ represents percent remaining and ▪ represents percent reduction. FIG. 5 is a plot of methyl mercaptan peak area vs. time obtained using Example 1A. ♦ represents percent remaining and ▪ represents percent reduction. FIG. 6 is a plot from a repeated time study using Example 1A. The plot shows methyl mercaptan peak area vs. time. ♦ represents percent remaining and ▪ represents percent reduction. FIG. 7 is a plot showing methyl mercaptan peak area vs. time obtained using Example 18. ♦ represents percent remaining and ▪ represents percent reduction. FIG. 8 is a plot showing methyl mercaptan peak area vs. time obtained using Example 1C. ♦ represents percent remaining and ▪ represents percent reduction. FIG. 9. is a plot showing methyl mercaptan peak area vs. time obtained using Example 1D. ♦ represents percent remaining and ▪ represents percent reduction. FIG. 10. is a plot showing methyl mercaptan peak area vs. time obtained using Example 1E. ♦ represents percent remaining and ▪ represents percent reduction.

With reference to FIGS. 2-10, it can be seen that the Example 1A and Example 1D samples had the greatest reduction in methyl mercaptan with ˜90% and 100% reduction, respectively. The remaining samples had the following methyl mercaptan reduction percentages: Example 1C (˜65%), Example 1B (˜54%), Example 1E (˜40%) and Comparative Example (˜25%). The data are listed in Table 4.

TABLE 4
Methyl Mercaptan Reduction Data.

| | | | | | | | Percentage | Percentage |
|---|---|---|---|---|---|---|---|---|
| | Time | Sample Peak | Control Peak | Control Peak | Control Peak | Control Peak | Remaining | Reduction |
| Sample | (Min) | Area | Area (Mercaptan) | Area (Average) | Area (Std. Dev.) | Area (RSD) | (Mercaptan) | (Mercaptan) |
| Example | 0.5 | 16308 | 22275 | 24661 | 1943 | 7.9% | 66% | 34% |
| 1A | 4.5 | 10595 | 22790 | | | | 43% | 57% |
| | 8.5 | 5837 | 26535 | | | | 24% | 76% |
| | 12.5 | 4638 | 24353 | | | | 19% | 81% |
| | 16.5 | 3361 | 25240 | | | | 14% | 86% |
| | 20.5 | 2417 | 28134 | | | | 10% | 90% |
| | 24.5 | 2366 | 23595 | | | | 10% | 90% |
| | 28.5 | 1872 | 24367 | | | | 8% | 92% |
| Example | 0.5 | 18670 | 22708 | 23227 | 586 | 2.5% | 80% | 20% |
| 1A | 4.5 | 11462 | 22960 | | | | 49% | 51% |
| | 8.5 | 7712 | 24586 | | | | 33% | 67% |
| | 12.5 | 5126 | 23248 | | | | 22% | 78% |
| | 16.5 | 4217 | 23299 | | | | 18% | 82% |
| | 20.5 | 3206 | 23065 | | | | 14% | 86% |
| | 24.5 | 2670 | 22801 | | | | 11% | 89% |
| | 28.5 | 2085 | 23151 | | | | 9% | 91% |
| Example | 0.5 | 20525 | 23870 | 24511 | 1099 | 4.5% | 84% | 16% |
| 1B | 4.5 | 16244 | 24106 | | | | 66% | 34% |
| | 8.5 | 14252 | 23700 | | | | 58% | 42% |
| | 12.5 | 12753 | 23901 | | | | 52% | 48% |
| | 16.5 | 12452 | 26983 | | | | 51% | 49% |
| | 20.5 | 11452 | 25208 | | | | 47% | 53% |
| | 24.5 | 10505 | 24133 | | | | 43% | 57% |
| | 28.5 | 11980 | 24183 | | | | 49% | 51% |
| Example | 0.5 | 22471 | 27752 | 25761 | 1658 | 6.4% | 87% | 13% |
| 1C | 4.5 | 15083 | 27913 | | | | 59% | 41% |
| | 8.5 | 12575 | 24816 | | | | 49% | 51% |
| | 12.5 | 10128 | 25313 | | | | 39% | 61% |
| | 16.5 | 10334 | 27102 | | | | 40% | 60% |
| | 20.5 | 9083 | 24925 | | | | 35% | 65% |
| | 24.5 | 9217 | 23193 | | | | 36% | 64% |
| | 28.5 | 8813 | 25077 | | | | 34% | 66% |
| Example | 0.5 | 15350 | 28784 | 26036 | 2229 | 8.6% | 59% | 41% |
| 1D | 4.5 | 3166 | 26167 | | | | 12% | 88% |
| | 8.5 | 794 | 24990 | | | | 3% | 97% |
| | 12.5 | 615 | 24647 | | | | 2% | 98% |
| | 16.5 | 213 | 24513 | | | | 1% | 99% |
| | 20.5 | 118 | 30174 | | | | 0% | 100% |
| | 24.5 | 0 | 24686 | | | | 0% | 100% |
| | 28.5 | 0 | 24328 | | | | 0% | 100% |
| Example | 0.5 | 16411 | 15640 | 15551 | 370 | 2.4% | 106% | −6% |
| 1E | 4.5 | 11253 | 15326 | | | | 72% | 28% |
| | 8.5 | 11621 | 15901 | | | | 75% | 25% |
| | 12.5 | 11230 | 15318 | | | | 72% | 28% |
| | 16.5 | 10506 | 15521 | | | | 68% | 32% |
| | 20.5 | 10766 | 14918 | | | | 69% | 31% |
| | 24.5 | 10934 | 16101 | | | | 70% | 30% |
| | 28.5 | 9137 | 15682 | | | | 59% | 41% |
| Comparative | 0.5 | 21521 | 23690 | 23908 | 914 | 3.8% | 90% | 10% |
| Example | 4.5 | 20019 | 24131 | | | | 84% | 16% |
| | 8.5 | 20298 | 25764 | | | | 85% | 15% |
| | 12.5 | 19490 | 23069 | | | | 82% | 18% |
| | 16.5 | 17400 | 24560 | | | | 73% | 27% |
| | 20.5 | 18096 | 23082 | | | | 76% | 24% |
| | 24.5 | 18148 | 23224 | | | | 76% | 24% |
| | 28.5 | 17381 | 23744 | | | | 73% | 27% |
| Comparative | 0.5 | 18074 | 25198 | 25189 | 1753 | 7.0% | 72% | 28% |
| Example | 4.5 | 21154 | 25667 | | | | 84% | 16% |
| | 8.5 | 21011 | 26323 | | | | 83% | 17% |
| | 12.5 | 19406 | 25381 | | | | 77% | 23% |
| | 16.5 | 18609 | 27451 | | | | 74% | 26% |
| | 20.5 | 19725 | 24691 | | | | 78% | 22% |
| | 24.5 | 18150 | 21377 | | | | 72% | 28% |
| | 28.5 | 18121 | 25426 | | | | 72% | 28% |

### Multi-Dosing Study in Aqueous Conditions

The following study is used to determined % headspace reduction (recovery) as described and claimed herein. Aqueous studies were performed in a manner that allows for the differentiation between the absorption and adsorption of methyl mercaptan by the liquid phase and silica, respectively. A multi-dosing protocol was used wherein repeated dosings of a sample slurry would allow for complete saturation with methyl mercaptan. By measuring the resultant headspace formation after each dosing and 60 minutes of equilibration, the adsorptive capacity of the samples in question can be determined indirectly.

Using the same apparatus shown in FIG. 1, the testing method was altered to reduce sources of error that could arise from inconsistent flask volumes, irregular evacuations and/or test gas dosing. The samples were run in a rotation, evaluating two samples at a time with the third flask acting as a blank control. For example, flask 1 would contain test slurry 1, flask 2 would contain test slurry 2, and flask 3 would contain the blank. After testing, the apparatus was reset with flask 1 containing the blank, flask 2 test slurry 1 and flask 3 test slurry 2. The final rotation would be flask 1 containing test slurry 2, flask 2 containing the blank, and flask 3 containing test slurry 1. The sample and control peak areas were averaged over all rotations, with the standard deviation and relative standard deviation (RSD) calculated to monitor the reliability of the test.

Using this method, the following samples were evaluated (200 mg sample, 800 distilled, deionized water): Example 1A, Example 1B, Example 1C, Example 1D, Comparative Example and Example 1E. FIG. 11 shows the resulting headspace recovery curves for each respective sample over repeated dosings with methyl mercaptan.

Based on this data, Example 1D has the greatest adsorptive capacity for methyl mercaptan, preventing headspace formation out to 6 dosings. Examples 1A, 1B and 1E show the next best performance out to 2 dosings before coalescing with Examples 1C and Comparative Example. The individual data points are listed in Table 5.

TABLE 5
Data from multi-dosing study.

| | | Rotation #1 | | Rotation #2 | | Rotation #3 | | | | Control | | |
|---|---|---|---|---|---|---|---|---|---|---|---|---|
| | # of | | Control | | Control | | Control | Sample | Control | Peak | Control | Head- |
| | dosings of | Sample | Peak | Sample | Peak | Sample | Peak | Peak | Peak | Area | Peak | space |
| | Methyl | Peak | Area | Peak | Area | Peak | Area | Area | Area | (Std. | Area | Recov- |
| Sample | Mercaptan | Area | (Mercaptan) | Area | (Mercaptan) | Area | (Mercaptan) | (Average) | (Average) | Dev.) | (RSD) | ery |
| Example | 1 | 499 | 4213 | 916 | 4586 | 68 | 4151 | 494 | 4317 | 235 | 5% | 11% |
| 1A | 2 | 2535 | 5127 | 3280 | 4870 | 1135 | 4713 | 2317 | 4903 | 209 | 4% | 47% |
| | 3 | 4059 | 5309 | 3782 | 4969 | 2690 | 4814 | 3510 | 5031 | 253 | 5% | 70% |
| | 4 | 4447 | 5318 | 3981 | 5279 | 3426 | 4703 | 3951 | 5100 | 344 | 7% | 77% |
| | 5 | 4586 | 5344 | 4067 | 4972 | 3697 | 4838 | 4117 | 5051 | 262 | 5% | 81% |
| | 6 | 4386 | 5121 | 4171 | 4972 | 3887 | 4850 | 4148 | 4981 | 136 | 3% | 83% |
| Comparative | 1 | 1493 | 4213 | 119 | 4586 | 1029 | 4151 | 880 | 4317 | 235 | 5% | 20% |
| Example | 2 | 3534 | 5127 | 1170 | 4870 | 3169 | 4713 | 2624 | 4903 | 209 | 4% | 54% |
| | 3 | 4434 | 5309 | 2576 | 4969 | 3754 | 4814 | 3588 | 5031 | 253 | 5% | 71% |
| | 4 | 4663 | 5318 | 3311 | 5279 | 3971 | 4703 | 3982 | 5100 | 344 | 7% | 78% |
| | 5 | 4893 | 5344 | 3474 | 4972 | 4102 | 4838 | 4156 | 5051 | 262 | 5% | 82% |
| | 6 | 4421 | 5121 | 3949 | 4972 | 4210 | 4850 | 4193 | 4981 | 136 | 3% | 84% |
| Example | 1 | 301 | 3515 | 473 | 4337 | 71 | 3670 | 282 | 3841 | 437 | 11% | 7% |
| 1B | 2 | 2574 | 3944 | 1906 | 4601 | 1355 | 4250 | 1945 | 4265 | 329 | 8% | 46% |
| | 3 | 3551 | 4169 | 3006 | 4649 | 2611 | 4344 | 3056 | 4387 | 243 | 6% | 70% |
| | 4 | 3588 | 4126 | 3611 | 4400 | 3176 | 4207 | 3458 | 4244 | 141 | 3% | 81% |
| | 5 | 4075 | 4445 | 3835 | 4521 | 3440 | 4118 | 3783 | 4361 | 214 | 5% | 87% |
| | 6 | 3822 | 3994 | 3557 | 4380 | 3425 | 3902 | 3601 | 4092 | 254 | 6% | 88% |
| Example | 1 | 1688 | 3515 | 227 | 4337 | 316 | 3670 | 744 | 3841 | 437 | 11% | 19% |
| 1C | 2 | 3418 | 3944 | 2185 | 4601 | 2138 | 4250 | 2580 | 4265 | 329 | 8% | 61% |
| | 3 | 4160 | 4169 | 3309 | 4649 | 2981 | 4344 | 3483 | 4387 | 243 | 6% | 79% |
| | 4 | 3796 | 4126 | 3534 | 4400 | 3264 | 4207 | 3531 | 4244 | 141 | 3% | 83% |
| | 5 | 4275 | 4445 | 3771 | 4521 | 3211 | 4118 | 3752 | 4361 | 214 | 5% | 86% |
| | 6 | 3984 | 3994 | 3380 | 4380 | 3466 | 3902 | 3610 | 4092 | 254 | 6% | 88% |
| Example | 1 | 0 | 3068 | 0 | 2906 | 0 | 3303 | 0 | 3092 | 200 | 6% | 0% |
| 1D | 2 | 0 | 3770 | 0 | 3714 | 0 | 3708 | 0 | 3731 | 34 | 1% | 0% |
| | 3 | 0 | 3945 | 0 | 3474 | 0 | 3635 | 0 | 3685 | 239 | 6% | 0% |
| | 4 | 0 | 4108 | 0 | 4025 | 0 | 2534 | 0 | 3556 | 886 | 25% | 0% |
| | 5 | 0 | 4323 | 0 | 3869 | 0 | 3806 | 0 | 3999 | 282 | 7% | 0% |
| | 6 | 0 | 4093 | 0 | 3632 | 0 | 3908 | 0 | 3878 | 232 | 6% | 0% |
| Example | 1 | 718 | 3068 | 200 | 2906 | 0 | 3303 | 306 | 3092 | 200 | 6% | 10% |
| 1E | 2 | 2718 | 3770 | 1738 | 3714 | 918 | 3708 | 1791 | 3731 | 34 | 1% | 48% |
| | 3 | 3327 | 3945 | 2762 | 3474 | 2118 | 3635 | 2736 | 3685 | 239 | 6% | 74% |
| | 4 | 3643 | 4108 | 3095 | 4025 | 2258 | 2534 | 2999 | 3556 | 886 | 25% | 84% |
| | 5 | 3530 | 4323 | 3489 | 3869 | 2569 | 3806 | 3196 | 3999 | 282 | 7% | 80% |
| | 6 | 3901 | 4093 | 3179 | 3632 | 2917 | 3908 | 3332 | 3878 | 232 | 6% | 86% |

### pH Ladder Study

The relationship between the adsorption of methyl mercaptan and the soluble metal species was observed. To determine at which point and the quantity of release, a pH ladder study was conducted. The method involved pH adjusting a slurry of the desired EXAMPLE to a specific pH, allowing equilibration, and analysis.

In a typical experiment, 1 gram of the desired Example material was suspended and stirred in a desired matrix and the solution pH adjusted using either 0.1M HCl or 0.1M NaOH. After the desired pH was reached, the slurry was allowed to equilibrate for 1 hour. The slurries were then centrifuged at 12,000 rpm for 10 minutes and the supernatant decanted. The supernatant was tested for the appropriate metal using ICP-OES.

Examples 1A-E were evaluated using this procedure, and the results are shown in FIG. 12.

Given that the 5% pH for these samples are all pH>8.0, there is little to no soluble metal ions present in solution. However, there is a significant release of soluble metal ions for Example 1A and Example 1D at around pH 5.0. This also corresponds to the low point of the Stephen curve, which maps the pH in the oral cavity over time after an initial carbohydrate introduction. The use of the Example 1A would allow for the release of Zn ions after a challenge to the oral cavity, thereby inhibiting bacterial growth or acting as an anti-microbial agent.

Various modifications and variations can be made to the compounds, composites, kits, articles, devices, compositions, and methods described herein. Other aspects of the compounds, composites, kits, articles, devices, compositions, and methods described herein will be apparent from consideration of the specification and practice of the compounds, composites, kits, articles, devices, compositions, and methods disclosed herein. It is intended that the specification and examples be considered as exemplary.

1. A method of reducing oral malodor, comprising administering an effective amount of a precipitated silica material having a mean particle size of 1 to 15 microns and comprising a metal adduct present on at least a portion of its surface, to the oral cavity of a mammal, thereby reducing oral malodor.

2. The method of claim 1, wherein the precipitated silica material exhibits at least a 35% headspace reduction in the Volatile Sulfur Compounds (VSC)s Absorption Test.

3. (canceled)

4. The method of claim 1, wherein the metal adduct comprises aluminum, zinc, tin, strontium, iron, copper, or a mixture thereof.

5-12. (canceled)

13. A precipitated silica material prepared by a process comprising: reacting a silicate with an acidulating agent and a metal salt, in a liquid medium, at a temperature of from about ambient temperature to about 130° C., to provide a reaction product in the liquid medium; and reducing the pH of the liquid medium to precipitate the silica material from the liquid medium; wherein the metal salt comprises a zinc(II) salt, a tin(II) salt, a iron(III) salt, a copper(II) salt, or a mixture thereof.

14. The precipitated silica material of claim 13, wherein the metal salt comprises zinc(II) chloride, tin(II) chloride, iron(III) nitrate, copper(II) nitrate, or a mixture thereof.

15. (canceled)

16. A precipitated silica material having a mean particle size of from 1 to 15 microns and comprising a metal adduct present on at least a portion of its surface; wherein the precipitated silica material exhibits at least a 35% headspace reduction in the Volatile Sulfur Compounds (VSC)s Absorption Test.

17. (canceled)

18. The precipitated silica material of claim 16, wherein the metal adduct comprises aluminum, zinc, tin, strontium, iron, copper, or a mixture thereof.

19-20. (canceled)

21. A dentifrice comprising:
(a) a precipitated silica material having a mean particle size of from 1 to 15 microns and comprising a metal adduct present on at least a portion of its surface; wherein:
the metal adduct comprises aluminum, tin, strontium, iron, copper, or a mixture thereof; and
the precipitated silica material exhibits at least a 35% headspace reduction in the Volatile Sulfur Compounds (VSC)s Absorption Test; and
(b) optionally at least one other component selected from at least one abrasive other than precipitated silica material, at least one thickening agent other than the precipitated silica material, at least one solvent, at least one preservative, and at least one surfactant, wherein the precipitated silica material is present as an abrasive agent, thickening agent, or both, within the dentifrice.

22. The dentifrice of claim 21, wherein the precipitated silica material exhibits at least a 50% headspace reduction in the Volatile Sulfur Compounds (VSC)s Absorption Test.

23. The dentifrice of claim 21, wherein the metal adduct is present in an amount ranging from about 1% to about 3% by weight of the precipitated silica material.

24. The dentifrice of claim 21, wherein the metal adduct comprises aluminum.

25. The dentifrice of claim 21, wherein the metal adduct comprises tin.

26. The dentifrice of claim 21, wherein the metal adduct comprises strontium.

27. The dentifrice of claim 21, wherein the metal adduct comprises iron.

28. The dentifrice of claim 21, wherein the metal adduct comprises copper.

29. The dentifrice of claim 28, wherein the metal adduct is present in an amount ranging from about 1% to about 3% by weight of the precipitated silica material.

30. The dentifrice of claim 21, wherein the metal adduct comprises:
aluminum and copper,
strontium and copper, or
tin and strontium.

31. The dentifrice of claim 30, wherein the metal adduct is present in an amount ranging from about 1% to about 3% by weight of the precipitated silica material.
